(19) United States
(12) Patent Application Publication
(10) Pub. No.: US 2004/0209239 A1
(43) Pub. Date: Oct. 21, 2004

(54) Methods and compositions fir identifying episomal dna virus infection treatment

(76) Inventor: Paul Lieberman, Wynnewood, PA (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 10/478,300

(22) PCT Filed: May 29, 2002
(86) PCT No.: PCT/US02/15150

Related U.S. Application Data
(60) Provisional application No. 60/296,193, filed on Jun. 6, 2001.

(51) Int. Cl. C12Q 1/70; C12Q 1/68

(52) U.S. Cl. 435/5

(57) ABSTRACT

Compounds useful in the treatment of infection by an episomal DNA virus are identified by a method involving the steps of (1) providing a culture of mammalian or avian cells containing multiple copies of a recombinant viral extrachromosomal episome having an episomal maintenance element, the episomes being associated with a detectable label; (2) contacting the culture with a test molecule that inhibits the interaction between the episomal maintenance element on the episomes and at least one enzyme selected from a family of enzymes consisting of the poly-ADP ribose polymerase family, the poly-ADP glycosylase family, the telomerase family, the myb DNA binding family that interferes with telomeres, and the telomere repeat binding protein family; and (3) detecting the elimination of the episomes from the cells by detecting the presence of the detectable label in the medium of the culture. These compositions are useful in pharmaceutical compositions for treating an infection by an episomal DNA virus and in diagnostic kits.

### BACKGROUND OF THE INVENTION

A variety of DNA viruses, particularly those of the Herpesviridae and Papillomaviridae families, reside within the host as episomes in a latent phase that follows active infection, disease and the recovery therefrom. As one example, Epstein-Barr virus, a herpes family virus, is maintained as an extrachromosomal multicopy episome in the nucleus of human B-lymphocytes and replicates once per cellular division cycle (Kieff, E., Epstein-Barr virus and its replication, in Field's Virology, Vol. 2 (eds. Knipe, D. & Howley, P. M.) 2343-2396 (Lippincott-Raven Publishers, Philadelphia, 1996); Rickinson, A. B. & Kieff, E. Epstein-Barr Virus, in Fields Virology, Third Edition 2397-2446 (Lippincott-Raven Publishers, 1996)).

EBV infects over 90% of the adult population worldwide and establishes a latent infection that persists for the life of the host. EBV acts as a cofactor in several malignancies, including Burkitt's lymphomna, Hodgkin's disease, nasopharygeal carcinoma and lymphoproliferative disease of immunosuppressed subjects, including transplant patients and patients with an X-linked genetic predisposition to such disease. EBV is also involved in AIDS lymphoma, some forms of gastric carcinoma, body cavity lymphoma, esophageal carcinoma T-cell lymphoma, some forms of breast cancer and in multiple sclerosis. In all instances it is the persisting latent virus that is detected in tumor biopsies or in associated disease tissues (e.g., CNS fluid for MS).

Latent cycle DNA replication initiates at the Dyad Symmetry (DS) region of the EBV origin of virus replication (OriP) and requires the viral encoded EBNA1 protein (Yates, J. L. et al, 1985 Nature 313, 812-815). The maintenance and replication of the EBV episomal viral genome during latency is dependent upon EBNA1 (Adams, A., 1987 J. Virol., 61:1743-1746). EBNA1 binds to multiple sites in two domains of OriP, the family of repeats (FR) and the DS element (Yates, J. L. and Guan N., 1991 J. Virol., 65:483-488). See FIG. 1A. The DS is the origin of DNA replication for EBV episomes and the FR is required for stable maintenance of the plasmid after the first several rounds of replication (Yates, J. L. et al, 1985 Nature, 313:812-815; Aiyar, A. et al, 1998 EMBO J., 17:6394-6403). The DS consists of four EBNA1 binding sites and three nonamer repeats which contribute to the replication efficiency and plasmid maintenance function of OriP (Gahn, T. A. & Schildkraut, C. L., 1989 Cell, 58, 527-535; and Frappier, L. & O'Donnell, M., 1992 J. Virol 66, 1786-1790). EBNA1 induces structural changes in the DS element and can link two regions of DNA through an oligomerization domain important for replication and plasmid maintenance function (Hearing, J. et al, 1992 J. Virol. 66; 694-705; Mackey, D. & Sugden, B., 1999 Mol. Cell. Biol. 19, 3349-3359; and Bochkarev A. et al, 1995 Cell, 83:39-46). Since EBNA1 lacks intrinsic helicase activity, it is thought to recruit cellular proteins required for initiation of DNA replication and for the stable maintenance of episomal DNA.

Latent infection of a host, particularly a human host, follows active infection with other similar DNA episomal viruses, such as human papilloma virus, human herpesviruses 6 through 8, cytomegalovirus, and varicella zoster virus. These latent viruses are similarly involved in diseases of their infected hosts.

There remains a need in the art for the identification and design of compounds which can effectively treat active and latent infection and ameliorate the diseases in which such viruses are involved.

### BRIEF SUMMARY OF THE INVENTION

In one aspect, the present invention involves a method for identifying a compound useful in the treatment of an infection by an episomal DNA virus. One embodiment of such a method comprising the steps of providing a culture of mammalian or avian cells containing multiple copies of a recombinant viral extrachromosomal episome having an episomal maintenance element. Each recombinant episome is associated with a detectable label. The culture is contacted with a test molecule that inhibits the interaction between the episomal maintenance element on the episomes and at least one selected enzyme. The enzyme may be a member of the poly-ADP ribose polymerase family, the poly-ADP glycosylase family, the telomerase family, the myb DNA binding family that interferes with telomeres, or the telomere repeat binding protein family. Elimination of the episomes from the cells is detected by observing the presence of detectable label in the culture medium.

In another aspect, the invention provides a kit for use in an assay for identifying a compound useful in the treatment of an infection by an episomal DNA virus. The kit contains a culture of the mammalian or avian cells containing multiple copies of the labeled recombinant viral extrachromosomal episome having an episomal maintenance element, as described above. The kit also contains some suitable means for detecting the presence of the detectable label in the culture medium following contact with a test molecule, as well as instructions for performing the assay.

In still another aspect, the invention provides a pharmaceutical composition comprising in a physiologically acceptable carrier, at least one compound that inhibits the interaction between the episomal maintenance element of a viral episome residing in the cells of a mammalian subject previously infected with an episomal DNA virus and at least one enzyme selected from the above-identified families of enzymes.

In yet a further aspect, the invention provides a method for treating an infection by an episomal DNA virus by administering to a mammalian subject having cells carrying multiple copies of a recombinant viral extrachromosomal episome having an episomal maintenance element an effective amount of at least one compound that inhibits the interaction between the episomal maintenance element on said episomes and at least one enzyme. The enzyme is selected from enzymes of the poly-ADP ribose polymerase family, the poly-ADP glycosylase family, the telomerase family, the telomere repeat binding protein family, and the myb DNA binding family that interferes with telomeres.

In still another aspect, the invention provides a method of increasing the cellular stability of a DNA molecule comprising an episomal maintenance element of an episomal DNA virus, said method comprising contacting said cell with an effective amount of a compound that inhibits the enzymatic activity of a member of the poly-ADP ribose polymerase enzyme family in the cell.

Other aspects and advantages of the present invention are described further in the following detailed description of the preferred embodiments thereof.

### BRIEF DESCRIPTION OF THE DRAWINGS

FIG. 1 is a schematic of EBV OriP structure consisting of the Family of Repeats (FR) and Dyad Symmetry (DS) region.

FIG. 2 is a schematic of the interaction between the DS, EBNA1 and cell factors.

FIG. 3 is a bar graph showing luciferase activity for the dominant negative deletion mutant of TRF2 in transient transfection assays.

FIG. 4 is a graph showing that a telomerase inhibitor VI, a 2-O-methyl oligonucleotide containing TTAGGG, disrupts plasmid maintenance of OriP.

FIG. 5A is a graph showing that a PARP inhibitor, 3-AB, enhanced the stability of the EBV genome in cells.

FIG. 5B is a graph showing that a PARP inhibitor, niacinamide, enhanced stability of the EBV genome in a cell. Treatment of cells with a DNA damaging agent alone, hydroxyurea, decreased the stability of the EBV genome in cells.

FIG. 5C is a graph showing that a PARP inhibitor, niacinamide, increased OriP maintenance in the cell. Treatment of the cells with a DNA damaging agent, hydroxyurea, decreased the maintenance of the OriP plasmid in the cell.

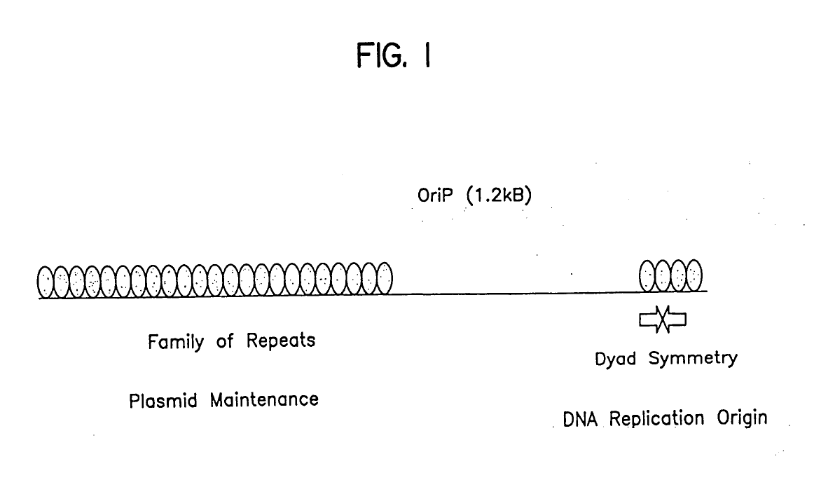

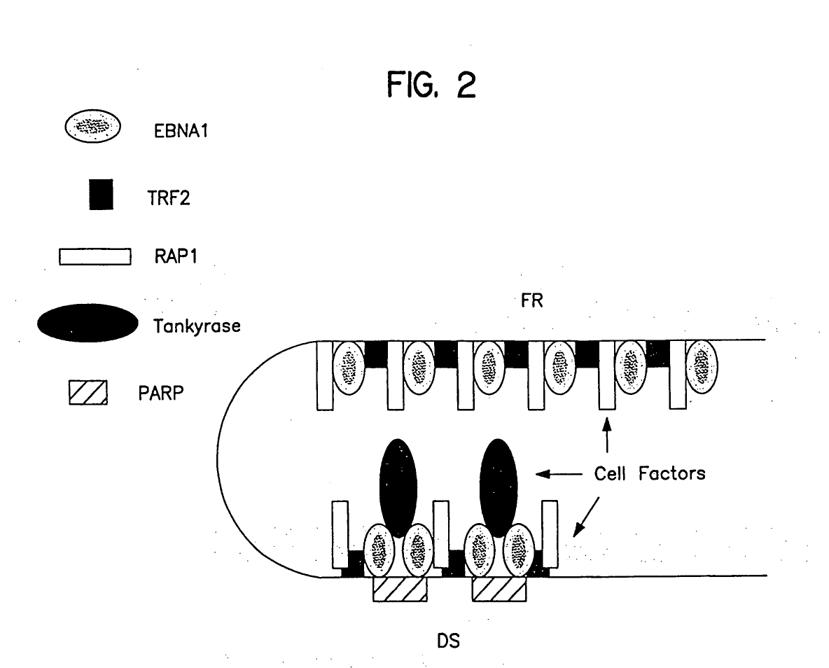

### DETAILED DESCRIPTION OF THE INVENTION

The present invention meets the needs in the art by providing a screening method for identifying a compound or molecule useful in the treatment of an infection by an episomal DNA virus, as well as compositions, kits and methods using these compounds for treating viral infections, in both the active and latent stages of infection. This invention is based upon the discovery that particular known families of enzymes and cellular proteins having a previously unknown function. Such enzymes and proteins identified below and useful in the methods of this invention were found to be associated with the maintenance of viral episomes in cells of infected hosts.

### I. Screening Method

The present invention provides a method for identifying a compound or molecule useful in the treatment of an infection by an episomal DNA virus by interaction with a selected cellular enzyme, resulting in destabilization of the episomal form of the virus in the infected cells. Briefly summarized, this method uses a culture of mammalian or avian cells containing multiple copies of a recombinant viral extrachromosomal episome having an episomal maintenance (EM) element. These recombinant episomes are associated with a detectable label. Contact of this cell culture with an effective test molecule causes loss of the episome from the cell. The detection of the expression of the label in the cell medium is indicative of loss of the viral episome from the cell.

A. Episomal DNA Viruses

Episomal DNA viruses for use in the present invention include primarily members of the Herpesviridae family, including, for example, Herpes Simplex virus (HSV), human Herpes virus 6 (HHV6), human Herpes virus 7 (HHV7), human Herpes virus 8 (HHV8), Cytomegalovirus (CMV), Epstein Barr virus (EBV), and Varicella zoster virus (VZV). Still another episomal DNA virus is a papillomavirus, such as human papillomavirus (HPV). An episomal DNA virus that infects fowl is the causative agent of Marek's disease. Following an active infection, these viruses reside episomally in the host in certain cell types, including epithelial cells, e.g., EBV and HBV; ganglia (e.g., VZV and HSV); monocytes (e.g., CMV), endothelial cells (e.g., HHV8) and lymphocytes, particularly B lymphocytes (e.g., EBV, HHV6 and HHV7). Subsequently, these viruses may be activated to cause a latent infection in the host, resulting in a variety of disorders.

In their episomal states, these viruses each contain an episomal maintenance (EM) element, which is a segment of between about 200 to 1000 nucleotides of viral DNA that is involved in maintaining and replicating the virus in the host cell. As an example, one such episomal maintenance element is a viral origin of replication, e.g., the oriP of EBV, the OriS or Ori L of HSV. Other episomal maintenance elements are referred to as terminal repeat (TR) sequences, such as those in HSV, HHV6, HHV7 and HHV8. HPV's episomal maintenance sequence is referred to as a long-control region (LCR). Still other terms for the EM elements include autonomous replicating sequence and matrix attachment region. The DNA sequences of EM sequences for these and other viruses may all be found published in Genbank.

B. Cell Cultures Containing the Recombinant Viral Episome

For use in the present invention, the recombinant viral episome requires, at a minimum, a selected episomal maintenance element of a selected episomal DNA virus, a detectable label, an optional trans-acting factor, and other optional sequences conventionally known to be useful in bacterial plasmids. For eukaryotic cells, such sequences useful in episomes or plasmids typically include a promoter (useful to express the label and/or an optional trans-acting factor), an enhancer, such as one derived from an immunoglobulin gene, SV40, cytomegalovirus, etc., a polyadenylation sequence which may include splice donor and acceptor sites and an optional intron sequence. One possible intron sequence is also derived from SV-40, and is referred to as the SV-40 T intron sequence. Another vector element that may be used is an internal ribosome entry site (IRES). An IRES sequence is used to produce more than one polypeptide from a single gene transcript. Selection of these and other common plasmid or vector elements are conventional and many such sequences are available (see, e.g., Sambrook et al, and references cited therein at, for example, pages 3.18-3.26 and 16.17-16.27 and Ausubel et al., “Current Protocols in Molecular Biology”, John Wiley & Sons, New York, 1989).

The episomal maintenance sequence may be isolated from cells infected with EBV, using conventional techniques such as by polymerase chain reaction amplification with primers based on its published sequence, and purification techniques, such as affinity chromatography. Alternatively, the EM sequence may be synthetically prepared from the published sequence by conventional sequence synthesis methods.

As mentioned above, still another component of the recombinant episome is a transacting factor of the selected virus. Among useful trans-acting factors are included, without limitation, EBNA-1 for EBV, E1 or E2 for HPV, UL9 for HSV, and Lana for HHV8. Preferably, the trans-acting factor is placed on the same plasmid as the EM element, but under the regulatory control of a selected promoter. Alternatively, the trans-acting factor is not on the same plasmid as is the EM, but is stably integrated into the cell. Useful promoters for the trans-acting factor include its naturally-occupant, promoter or any other promoter, including one of those described below. Sequences for such trans-acting factors are known and may be found in Genbank or known and readily available publications on the relevant virus.

One or more labels are employed which provide one or more detectable signals, either directly or indirectly. The label may be located anywhere on the plasmid or episome. For instance, the label may be a fluorescent label, a luminescent label, a radiolabel, or a chemiluminescent label. Such labels may preferably be reporter genes, which upon expression produce detectable gene products. Such reporter sequences include without limitation, DNA sequences encoding a lux gene, β-lactamase, a galactosidase enzyme, e.g., β-galactosidase (LacZ), alkaline phosphatase, thymidine kinase, green fluorescent protein (GFP), chloramphenicol acetyltransferase (CAT), a luciferase enzyme, or a gluconase enzyme. Still other labels which may be attached to the EM sequence include membrane bound proteins including, for example, CD2, CD4, CD8, the influenza hemagglutinin protein, a biotin molecule and others well known in the art, to which high affinity antibodies directed thereto exist or can be produced by conventional means. Still other detectable labels include fusion proteins comprising a membrane bound protein appropriately fused to an antigen tag domain from, among others, hemagglutinin or a Myc gene. Still other detectable labels may include hybridization or PCR probes. Any number of additional, and conventionally employed, label systems may be adapted to the method of this invention. One of skill understands that selection and/or implementation of a label system involves only routine experimentation.

If the label and/or the trans-acting factor requires a promoter for expression, the expression control sequences—native, constitutive, inducible and/or tissue-specific—may be selected from among those known in the art. The following non-exclusive list of promoters may be utilized to drive expression of the label and/or the trans-acting factor, depending upon the type of expression desired. Examples of high-level constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, 1985 Cell, 41:521-530), the SV40 promoter, the dihydrofolate reductase promoter, the β-actin promoter, the phosphoglycerol kinase (PGK) promoter, and the EF1α promoter (Invitrogen). Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, 1996 Proc. Natl. Acad. Sci. USA, 93:3346-3351), the tetracycline-repressible system (Gossen et al, 1992 Proc. Natl. Acad. Sci. USA, 89:5547-555 1), the tetracycline-inducible system (Gossen et al, 1995 Science, 268:1766-1769; see also Harvey et al, 1998 Curr. Opin. Chem. Biol., 2:512-518), the RU486-inducible system (Wang et al, 1997 Nat. Biotech., 15:239-243 and Wang et al, 1997 Gene Ther., 4:432-441) and the rapamycin-inducible system (Magari et al, 1997 J. Clin. Invest., 100:2865-2872). Other types of inducible promoters which may be useful in this context are those which are regulated by a specific physiological state, e.g., temperature, acute phase, a particular differentiation state of the cell, or in replicating cells only.

In another embodiment, the native promoter for the trans-acting factor is used. The native promoter may be preferred when it is desired that expression of the trans-acting factor should mimic the native expression. The native promoter may be used when expression of the trans-acting factor must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of a promoter for the label or trans-acting factor includes a tissue-specific promoter. For instance, if expression in skeletal muscle is desired, a promoter active in muscle should be used. These include the promoters from genes encoding skeletal α-actin, myosin light chain 2A, dystrophin, muscle creatine kinase, as well as synthetic muscle promoters with activities higher than naturally-occurring promoters (see Li et al., 1999 Nat. Biotech., 17:241-245). Examples of promoters that are tissue-specific are known for liver (albumin, Miyatake et al. 1997 J. Virol., 71:5124-32; hepatitis B virus core promoter, Sandig et al., 1996 Gene Ther., 3:1002-9; alpha-fetoprotein (AFP), Arbuthnot et al., 1996 Hum. Gene Ther., 7:1503-14), bone (osteocalcin, Stein et al., 1997 Mol. Biol. Rep., 24:185-96; bone sialoprotein, Chen et al., 1996 J. Bone Miner. Res., 11:654-64), lymphocytes (CD2, Hansal et al., 1998 J. Immunol., 161:1063-8; immunoglobulin heavy chain; T cell receptor α chain), neuronal (neuron-specific enolase (NSE) promoter, Andersen et al. 1993 Cell. Mol. Neurobiol., 13:503-15; neurofilament light-chain gene, Piccioli et al., 1991 Proc. Natl. Acad. Sci. USA, 88:5611-5; the neuron-specific vgf gene, Piccioli et al., 1995 Neuron, 15:373-84); among others.

Conventional techniques may be utilized for construction of the recombinant sequences comprising the labeled EM sequences of the invention. For example, the nucleotide sequences of the invention may be prepared conventionally by resort to known chemical synthesis techniques, e.g., solid-phase chemical synthesis, such as described by Merrifield, 1963 J. Amer. Chem. Soc., 85:2149-2154, and J. Stuart and J. Young, Solid Phase Peptide Synthesis, Pierce Chemical Company, Rockford, Ill. (1984). Alternatively, the nucleotide sequences of this invention may be prepared by known recombinant DNA techniques and genetic engineering techniques, such as polymerase chain reaction, by cloning within a host microorganism, etc. (See, e.g., Sambrook et al., cited above; Ausubel et al. (1997), Current Protocols in Molecular Biology, John Wiley & Sons, New York).

For example, the selected EM sequence and preferred label sequence may be obtained from commercial sources (e.g., Invitrogen) or from gene banks derived from whole genomic DNA. These sequences, fragments thereof, modifications thereto and the full-length sequences are preferably constructed recombinantly using conventional molecular biology techniques, site-directed mutagenesis, genetic engineering or PCR, and the like by utilizing the information provided herein. For example, methods for producing the above-identified modifications of the sequences include mutagenesis of certain nucleotides and/or insertion or deletion of nucleotides are known and may be selected by one of skill in the art.

The preparation or synthesis of the nucleotide sequences disclosed herein, whether in vitro or in vivo (including ex vivo) is well within the ability of the person having ordinary skill in the art using available material. The synthetic methods are not a limitation of this invention. The examples below detail presently preferred embodiments of synthesis of these sequences. The labels are coupled or fused to the EM nucleotide sequence by conventional means, suitable for the particular label. See, generally, Sambrook et al, cited above.

Once the desired recombinant episomes are engineered, they may be transferred to a selected mammalian or avian host cell by any suitable method. Such methods include, for example, transfection, electroporation, liposome delivery, membrane fusion techniques, high velocity DNA-coated pellets, infection and protoplast fusion.

Suitable mammalian cells include, without limitation, epithelial cells, endothelial cells, ganglion, lymphocytes, preferably B lymphocytes, monocytes. Examples of such cells include CHO, BHK, MDCK, and various murine cells, e.g., 10T1/2 and WEH1 cells, African green monkey cells, suitable primate cells, e.g., VERO, COS1, COS7, BSC1, BSC 40, and BMT 10, and human cells such as W138, MRC5, A549, human embryonic retinoblast (HER), human embryonic kidney (HEK), human embryonic lung (HEL), TH1080 cells. Other suitable cells may include N1H3T3 cells (subline of 3T3 cells), HepG2 cells (human liver carcinoma cell line), Saos-2 cells (human osteogenic sarcomas cell line), HuH7 cells or HeLa cells (human carcinoma cell line). In a preferred embodiment, appropriate cells include the human embryonic kidney 293T cells (which express the large T antigen) (ATCC). Suitable avian cells include, without limitation, chicken cells, such as DT40 B lymphocytes. Neither the selection of the mammalian or avian species providing the host cells nor the type of cell is a limitation of this invention.

In yet another embodiment of this invention, the host cells include cells from a mammalian or avian species, which are previously naturally infected. These cells may be useful in the following methods if transfected with a suitable plasmid containing a label that will be detectable as expelled from the infected host with the viral episome.

In either case, whether the cells are transfected with recombinant episomes or the viral episomes occur in infected cells of infected hosts, the cells are then cultured in media suitable for the support of the mammalian or avian cells, as indicated, and this culture of cells is employed in the present screening method. The cells in the culture are generally in a growth medium suitable to the selected cells. There are any number of commercially available growth media which may be selected for the cell type. It should be understood that selection of a suitable medium is well within the skill of the art. See, e.g., commercial media catalogs such as those produced by Difco and BBL, as well as the media described in texts, such as MICROBIOLOGY, INCLUDING IMMUNOLOGY AND MOLECULAR GENETICS, 3rd edit, Davis et al, eds., Harper & Row Publ., Philadelphia 1980; and MEDICAL MYCOLOGY, J. W. Rippon, Ph.D., edit, W. B. Saunders Co, Philadelphia 1974, among others.

C. The Test Compound or Molecule

According to this method, the above-described cell culture is contacted with a test molecule which is assayed for the ability to interact with a selected cellular enzyme and eliminate the recombinant episome from the cell.

By the term “test molecule” is meant any synthetic, recombinantly-produced or naturally-occurring protein, including an antibody, or a small peptide and, alternatively, any synthetic or naturally-occurring chemical compound, including compounds contained or produced in combinatorial libraries, or compounds for which the structures were designed by computer or three dimensional analysis. Generally, pharmaceutical companies have batteries of unknown or undeveloped compounds which may be “test compounds” for purposes of this invention.

Such test compounds may be used in the method of this invention alone, in an inert buffer, e.g., saline, or may contain a solvent. Suitable solvents for test compounds may be readily selected by the art from standard chemistry texts, and include dimethylsulfoxide (DMSO), alcohols, such as methanol and ethanol, or aqueous solutions, such as water, culture medium, etc. Other useful solvents may be selected from among those known in the art (See, e.g., other solvents discussed in ORGANIC CHEMISTRY, 3rd edit., R. Morrison et al, eds., Allyn and Bacon, Inc., Boston, Mass. 1973 and MEDICINAL CHEMISTRY, 2nd edit., T. Nogrady edit., Oxford University Press, New York 1988). The test compound is selected for its ability to inhibit the interaction between the episomal maintenance element and at least one, and preferably more than one, cellular enzyme.

The cellular enzyme with which the test molecule interacts may be a member of the poly-ADP ribose polymerase (PARP) family of enzymes. PARP enzymes, including the Tankyrase enzyme, function as DNA damage checkpoint proteins. Poly-ribosylation is a modification of proteins that have rapid turnover. These enzymes associate with the EM sequence and ADP ribosylate other proteins, e.g., EBNA1. Tankyrase binds TRF1 and is associated with the EBV EM sequence. Tankyrase has PARP activity. Thus, inhibitors of PARP are likely to inhibit tankyrase. Like PARP, tankyrase functions as a DNA damage checkpoint protein. Inhibitors of Tankyrase in the presence of DNA damaging agents may preferentially mutate and inactivate the viral genomes. The PARP inhibitors, 3-aminobenzamide and niacinamide, weakly inhibit transient replication of episomal plasmids, but stabilize long term maintenance of such plasmids. The inventors have discovered that inhibitors of these enzymes, in the presence of DNA damaging agents, preferentially mutate and inactivate viral genomes.

Alternatively or additionally, the cellular enzyme may be a member of the poly-ADP glycosylase (PARG) family. PARG enzymatically removes the poly-ADP ribose moiety from modified proteins; thus these enzymes are important for maintenance of the viral genome during latency. PARG inhibitors have been determined by the inventor to cause a destabilization of the latent viral genome, opposing the effects found for PARP inhibitors in the absence of genotoxic stress.

Alternatively or additionally, the cellular enzyme may be a member of the telomerase family. Telomerase specifically associates and functions at the EM sequence of these viruses, and is important for plasmid maintenance. Telomerase inhibitors interfere with viral plasmid maintenance and are candidate highly effective compounds for inhibiting latent cycle replication and plasmid maintenance of such viruses. Modified telomerase enzymes may be involved in viral replication and plasmid maintenance.

A smaller telomerase-related polypeptide (60 kDa) has been found by the inventor to be associated with the EM sequence of EBV. This smaller polypeptide has telomerase activity, but may lack some of the processivity associated with full length telomerase. This polypeptide cross-reacts with Tert, a catalytic subunit of telomerase. This smaller version of telomerase is anticipated to have unique properties distinct from full length telomerase that make it a novel target for disruption in conditions caused by these DNA viruses, e.g., EBV associated tumors.

Still another family of enzymes that may be inhibited by the test molecule to achieve its effect is the telomere repeat binding factor or protein (TRF) family. The telomere repeat binding protein family contains, without limitation, telomere repeat binding protein 1, telomere repeat binding protein 2, and human RAP1. The TRF1 and 2 bind directly to the EM sequence of EBV. At telomeres, these enzymes bind to and stabilize quadruplex DNA structures. Quadruplex DNA analogs have been shown to inhibit telomere length stability. Any compound that interferes with TRF1 and TRF2 function is likely to inhibit DNA virus latent cycle replication and stability.

D. Steps of the Screening Method

In one embodiment of the screening method of this invention, a test compound or molecule is contacted with the above-described mammalian or avian cell culture.

The culture may be grown in any typical laboratory equipment, e.g., a multi-well plate. The total volume for each well used in the screening assay of the invention comprises generally the cell culture (the cells or other particles and culture medium) and the amount of test compound (including any amount of solvent contained therein) added. The total volume of cell culture and test compound is generally present in a standard dilution or ratio. Generally the ratio of the compound to cell culture is limited only by the identity and amount of the solvent, if any, in which the test compound is present and upon the concentration of the test compound that is desired.

Generally, the test compound is used at a concentration ranging from about 0.1 μM to about 10 μM, although higher and lower concentrations may be employed. Other concentrations may be used depending upon the potency of the compound's effect on the selected cells used in the assay. If the compound is in any solvent (e.g., DMSO) other than the culture broth, the concentration of the solvent in the final reaction mixture is preferably limited to about 5% of the total volume. More preferably, the solvent concentration, if any, in the test compound is less than about 2% of the total volume of the well.

As stated above, the ratio of test compound to cell culture can be any standard dilution, such as 1:50. For example, it is possible to dilute the compound between ratios of about 1:500 to about 1:5, as long as the compound is in the appropriate solvent (e.g., in the case of 1:5 dilution, the compound must be in growth medium), and is appropriately concentrated. Still other ratios can be used. In a preferred embodiment, the total volume of cell culture and test compound added to each well in a standard 1:50 dilution is generally about 1 μL of 10 μM compound or less for every 49 μL of cell culture in the wells.

In the practice of this screening assay, preferably the cell culture is placed in the medium and grown at least for about 8 to 12 hours (i.e., “overnight”) at a suitable temperature for normal growth of that cell type. Such normal growth temperatures may be readily selected based on the known growth requirements of the selected culture. Preferably, during the establishment of the culture and particularly during course of the method, the cell culture is incubated in a controlled humidity suitable for growth of the selected cells before and after contact with the test compound. The humidity of the incubation is controlled to minimize evaporation from the microtiter vessel, and permit the use of small volumes. Alternatively, or in addition to controlling humidity, the vessels may be covered with lids in order to minimize evaporation. Selection of the incubation temperature depends upon the identity of the cells, primarily. Selection of the percent humidity to control evaporation is based upon the selected volume of the vessel and concentration and volume of the test compound and cell culture in the vessel, as well as upon the incubation temperature. Thus, the humidity may vary from about 10% to about 80%. It should be understood that selection of a suitable incubation temperature, and time of incubation prior to initiation of the assay method and selection of controlled humidity is well within the skill of the art. See the texts cited immediately above. The cells, once incubated, are preferably diluted to a suitable cell number, as described is above, in the same medium.

The test compound may be added to the cell culture immediately after the culture is established in an initial incubation, or the test compound may be added to the cell culture at a selected time during the course of the assay. Further, according to this assay, the plates with both test compound and cell culture are incubated at a desired temperature, which in one embodiment of this method, is the same initial incubation temperature for the cell culture. Alternatively, the incubation temperature may be varied during the course of the assay method, as desired to observe temperature fluctuation effects upon the test compound and cell culture collectively or individually. As the temperature fluctuates in this embodiment, the humidity may be adjusted to prevent evaporation.

In one variant of the assay of this invention, each well may contain a single cell culture contacted with a single test compound. In another variant, each well may contain multiple different test compounds in the desired dilution. Another alternative is that each well may contain more than a single type of cell and each well is contacted with a different test compound, or with multiple test compounds.

According to one embodiment of this invention, the plates containing test compound and culture are removed from incubation and the effect of the test compound is detected at one timepoint or at multiple timepoints during the growth of the culture, after the culture has been in contact with the compound. Alternatively, such detection can occur while the cell is in contact with the compound. In one embodiment, the assay involves removing the test compound from contact with the culture at a selected time after said culture has been in contact with said test compound and before detection of the desired effect. Such removal may be effected by rinsing the cells gently or by some other gentle procedure. Still other embodiments involve repeatedly contacting the culture with one or more test compounds, and/or repeatedly removing the test compounds prior to detection, or contacting the culture with increasing dosages of the test compounds. Still another modification of the screening assay includes adding additional components, e.g., DNA damaging agents, to the culture so that more than a single effect may be detected by the assay.

Whatever variant of well contents is selected for detection, it may be preferred to shake the wells prior to each detecting step. With regard to the detection of the desired effect, more than a single detection or type of detection may be employed to detect the effect(s) of the test compound(s) on the culture. The detecting steps may be repeated for multiple different test compounds on the same culture, or repeated for the same test compounds on multiple different cultures. The assay may repeat the same or different detecting steps for multiple different test compounds on the same culture. Alternatively the assay may include repeating the same or different detecting steps for the same test compounds on multiple different cultures.

In one embodiment, a single detection is taken using this assay. More preferably, the total amount of time that the assay is performed is over a 24-hour growth period. The number of timepoints at which the test compound effect is preferably detected is at least two, including an initial detection at timepoint 0. The intervals between the detection of the effect may be regular, evenly spaced intervals. Alternatively, the intervals may be unevenly spaced intervals.

A desirable test compound is identified by its ability to mediate the elimination of the recombinant episomes from the cells, such as by detecting the presence of detectable label in the medium of the culture, rather than solely in the cells. The detecting method utilized must be compatible with the nature of the label itself. The means of label or reporter detection depend upon the identify of the label to which the EM sequence is attached in the recombinant episome. Such means of detection include, without limitation, enzymatic, radiographic, calorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the label is the LacZ gene, assays for beta-galactosidase activity are used to detect expression of the label. Where the selected label is luciferase, its expression by the synthetic sequence of the present invention may be detected by light production in a luminometer.

A suitable means of detection determines if the contact between the test compound and the cell culture inhibited the association of the EM element with one or more than one of the cellular enzymes. If so, that compound is identified as a desirable compound for use in the following methods of this invention. Preferably, the test molecule inhibits the association by binding directly to one or more than one of the above-described enzymes. Alternatively, the test molecule inhibits the association of the EM sequence with the cellular enzymes by preventing the binding of one or more of these enzymes to the EM sequence in a indirect manner, e.g., by interfering with the activity of other molecules necessary to effect proper binding between the EM sequence and one or more of the above-noted enzymes.

The method of the invention may also involve additional method steps, such as a step of assessing the identified “useful” test compound for toxicity to selected target cells. For example where the target cells are anticipated to be human cells, the identified test compound may be subjected to assays showing that target cell viability and/or growth and replication are not inhibited in the presence of test molecule. Assays that measure mitotic index, apoptosis and necrosis are useful for this purpose Suitable assay formats may be readily selected by one of skill in the art and additionally include assays for cell death and/or assays demonstrating the uptake of viral dyes, e.g., trypan blue. Such assay formats may readily be found by one of skill in the art in conventional laboratory manuals, such as CELLS, published by Cold Spring Harbor Laboratory.

In still another variation, this method can be employed to identify those test compounds which are found to enhance the stability of the recombinant episome in the cell. For example, test compounds which do not result in loss of episome from the cell, but rather increase the duration of episomal stability relative to no treatment at all, will have uses in gene therapy, as discussed below.

Yet another variation of this test molecule identification method involves exposing the cell cultures identified above to an agent of genotoxic stress, either before, during or after the time that the test compound is contacting the cells. Preferably, the agent of genotoxic stress is used at the same time that the cells are contacted with the test molecule. This method allows the identification of test molecules that have an appreciable effect of episomal elimination only in the presence of such agents.

There are a wide variety of agents of genotoxic stress, including, without limitation, ionizing radiation, such as gamma irradiation, or methylating agent and/or oxidizing agents, as well as other agents which inhibit DNA repair. For example, when radiation is used as the agent of genotoxic stress, the cell culture to which the test molecule has been added is exposed to gamma radiation at levels above 2-8 Gy. After sufficient time in culture, the presence of label in the medium is detected by the means described above. Alternatively, methylating agents, such as cis-platin, at doses above about 10 ng/ml per cell, are added to the cell culture with the test compound. After a suitable time of exposure to both the test molecule and the agent, e.g., up to about 1 hour, the culture is examined for the presence of label in the medium. In another embodiment, an oxidizing agent, such as hydrogen peroxide, is added to the cell culture with the test compound. After a suitable time of exposure to both the test molecule and the agent, the culture is examined for the presence of label in the medium. Still another genotoxic agent is ethidium bromide, which may be used at conventional levels to stress the cell culture. Selection of the genotoxic agent, as well as suitable dosages and times of exposure may be readily selected by one of skill in the art, taking into consideration the size and condition of the cell culture, as well as the identity of the cell type and virus.

Use of the above-described screening methods thus enables the identification of test molecules that may be useful in the treatment of infections by an episomal DNA virus, particularly infections of mammalian hosts, especially human hosts, as well as compounds useful in gene therapy.

### II. Kits

A kit for use in an assay for identifying test compounds useful in the treatment of infections by an episomal DNA virus or in identifying compounds useful in gene therapy desirably contains a culture of mammalian or avian cells containing multiple copies of a recombinant viral extrachromosomal episome having an episomal maintenance element, the episome being associated with a detectable label, as described above. Also present in the kit are one or more genotoxic agents, e.g., methylating or oxidizing agents. Means for detecting the presence of the detectable label in the medium of said culture following contact with a test molecule are also provided, based on the identity of the label. Additional components of such a kit include instructions for performing the identifying assay with or without the use of genotoxic agents, as well as optional instructions for performing a toxicity assay.

Such kits may contain one or more recombinant molecule or host cell of this invention, in free or immobilized form. The kits also include instructions for performing the specified assay, microtiter plates to which the molecules or nucleic acid sequences of the invention have been pre-adsorbed, various diluents and buffers, labeled conjugates for the detection of specifically bound compositions and other signal-generating reagents, such as enzyme substrates, cofactors and chromogens. Other components may include indicator charts for colorimetric comparisons, disposable gloves, decontamination instructions, applicator sticks or containers, and a sample preparator cup.

### III. Compositions Containing the Text Molecule and Methods of Use

In another embodiment of this invention, a pharmaceutical composition is provided that contains in a physiologically acceptable carrier, at least one compound that inhibits the interaction between the episomal maintenance element of a viral episome residing in the cells of a mammalian subject previously infected with an episomal DNA virus and at least one cellular enzyme as identified above. Test compounds that inhibit interaction between the EM sequence and one or more cellular enzymes and results in the loss of episomes from the subject's cells, without causing undue side effects to the subject, are desirably used in pharmaceutical compositions designed for treatment of infection of a mammalian host, preferably a human, with one of more of the episomal DNA viruses identified herein.

In one embodiment, the composition includes an inhibitor of PARP, such as 3-aminobenzamide or niacinamide or other nicotinic acid analogs. In another embodiment, the composition includes an inhibitor of PARG, such as a gallotannin or a tannic acid derivative. Still another embodiment includes a test compound that inhibits a telomerase or telomerase-related polypeptide.

The compositions may also contain a DNA damaging agent, such as a methylating agent or an oxidizing agent or other assent inhibiting DNA repair in an amount suitable to complement and enhance the activity of the test compound.

The composition desirably includes a carrier or excipient. Suitable carriers may be readily selected by one of skill in the art in view of the indication for which the test molecule is used. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the test molecule, other conventional pharmaceutical ingredients, such as preservatives, chemical stabilizers, or adjuvants. The pharmaceutical compositions may also contain other additives suitable for the selected mode of administration of the composition. Thus, these compositions can contain additives suitable for administration via any conventional route of administration, including without limitation, parenteral administration, intraperitoneal administration, intravenous administration, intramuscular administration, subcutaneous administration, intradermal administration, oral administration, topical administration, intranasal administration, intra-pulmonary administration, rectal administration, vaginal administration, and the like. All such routes are suitable for administration of these compositions, and maybe selected depending on the agent used, patient and condition treated, and similar factors by an attending physician. See, e.g., Remington: The Science and Practice of Pharmacy, Vol. 2, 19^{th} edition (1995), e.g., Chapter 95 Aerosols; and International Patent Application No. PCT/US99/05547, the teachings of which are hereby incorporated by reference. Routes of administration for these compositions may be combined, if desired, or adjusted.

Thus, such compositions may be used to treat infection of a mammal with an episomal DNA virus, such as a member of the Herpesviridae family, e.g., diseases caused by active or latent infection with Herpes Simplex virus, human Herpes virus 6, human Herpes virus 7, human Herpes virus 8, Cytomegalovirus, Epstein Barr virus, and Varicella zoster virus. Similarly such compositions may be designed for veterinary use, such as in the treatment of fowl with Marek's disease. The composition may also be employed to treat an active or latent infection of a host, e.g., a human, with a papillomavirus. Such a treatment method involves administering to a mammalian or avian subject having cells carrying multiple copies of a viral extrachromosomal episomes having an episomal maintenance element an effective amount of at least one compound or composition as described above that inhibits the interaction between the episomal maintenance element and at least one of the above-identified cellular enzymes. Preferably, the result of such inhibition is loss of the episomes from the subject's cells, without causing undue side effects to said subject.

An optional step of the method of treatment includes administering to said subject a DNA damaging agent, simultaneously or concurrently with the compound or composition described above. That DNA damaging agent may include one or more of gamma irradiation, a methylating agent or an oxidizing agent, or those agents mentioned above. These agents of genotoxic stress may be administered at known dosages and route of administration for the use in treating other diseases, such as cancer.

In general, the pharmaceutical compositions are administered in an amount effective to treat or prevent the diseases, disorders or infections for which it is designed. The amount of the pharmaceutical composition in a dosage unit employed will be determined empirically, based on the response of cells in vitro and response of experimental animals to the compositions of this invention. It will be appreciated that optimum dosage will be determined by standard methods for each treatment modality and indication. Thus the dose, timing, route of administration, and need for readministration of these compositions may be determined by one of skill in the art, taking into account the condition being treated, its severity, complicating conditions, and such factors as the age, and physical condition of the mammalian subject, the employment of other active compounds, and the like.

### IV. Methods of Increasing Stability of a DNA Molecule

In still another embodiment, the invention provides a method of increasing the cellular stability of a recombinant DNA molecule that comprises an episomal maintenance element of an episomal DNA virus. Preferably, such a DNA molecule is a recombinant, non-naturally occurring molecule, such as a DNA plasmid. Even more preferably, the DNA molecule is a gene therapy vector, based on a recombinant virus. Many such viral vectors are known, including recombinant retroviruses, adenoviruses, adeno-associated viruses, vaccinia viruses, etc.

This method includes contacting the cell transfected or infected with the DNA molecule with an effective amount of a compound that inhibits the enzymatic activity of a member of the poly-ADP ribose polymerase (PARP) enzyme family, including Tankyrase, in the cell, but does not eliminate viral episomes in the absence of additional genotoxic stress. PARP and tankyrase inhibitors stabilize the viral plasmids and may be used to increase the stability of plasmids used in gene therapy research and treatment methods. This class of compounds identified by one embodiment of the screening methods above, while they may weakly inhibit transient replication of the DNA molecules, has the additional use of stabilizing the long term maintenance of such DNA molecules or plasmids in mammalian cells, when used without any agents that place the cell under genotoxic stress. Thus, these compounds may be used to maintain plasmid stability in in vitro cell cultures. These compounds used in this method may also provide an in vitro treatment to assist gene therapy subjects in maintaining the vectors in their target cells.

In the in vitro method, a suitable amount of the compounds is added to the mammalian cell culture in question, e.g., >LD₅₀, following transfection or infection with the DNA molecule. In the in vivo method, a suitable amount of the compounds in a pharmaceutical composition such as described above may be administered to mammalian subjects concurrently or simultaneously with the DNA molecule to stabilize the molecule in the cells of the subjects. Such use enhances the stability of such vectors in transfected or infected cells and increase the time during which the vectors accomplish their therapeutic intent.

### V. The Examples

As shown in the Examples 1-6 below, DS specific DNA affinity chromatography and mass spectrometry were used to isolate and identify cellular proteins that bind to the Dyad Symmetry element of EBV oriP in the presence of EBNA1. The inventors have determined that a common set of proteins bind DS and participate in OriP-dependent episomal maintenance and telomere-dependent chromosome stability. These proteins participate in one or more of the following functions: initiation of viral DNA replication, plasmid maintenance, DNA damage recognition. These proteins, which also have known functions in telomere-length maintenance and chromosome stability, included: Telomeric Repeat Binding Factors (TRF) 1 and 2 (Broccoli, D. et al, 1997 Nat. Genet. 17, 231-235), TRF2-interacting protein hRAP1 (Li, B. et al, 2000 Cell 101, 471-483), and the TRF1-interacting Tankyrase (Smith, S. et al, 1998 Science 282, 1484-1487) bound to the DS element in an EBNA1-dependent manner. TRF2 and Tankyrase associate with OriP in vivo and can be recruited to the DS in an EBNA1-specific manner. It is likely that RAP1, TRF1, and PARP are similarly associated with OriP in vivo. These telomeric binding proteins regulate EBNA1-dependent replication of OriP.

TRF2 bound the nonamer sequence of OriP which has been shown genetically to contribute to DNA replication and plasmid maintenance function. TRF1 and TRF2 bind the mammalian telomeric repeat sequence (TTAGGG) and regulate telomere length. TRF2 associates with the MRE11-RAD50-EBNA1 facilitated recombinant double strand break repair complex and may prevent telomere ligation and recombination (Zhu, X.-D. et al, 2000 Nat. Genet., 25:347-352). TRF2 recognition of the DS nonamer repeats (TTAGGGTT) which resemble human telomeric repeats. Chromatin immunoprecipitation assay revealed that TRF2 and Tankyrase bound to episomal OriP in RAJI Burkitt lymphoma cells latently infected with EBV. Dominant negative mutants of TRF1 and TRF2 inhibited OriP-dependant DNA replication, indicating that these telomeric binding factors contribute to functions important for viral latency. These findings suggest that a common mechanism regulates the replication and stability of human telomeres and EBV episomes.

Tankyrase was isolated in a two-hybrid screen with TRF1, and consists of a large ankyrin repeat domain and a region homologous to PARP. PARP is a DNA damage recognition protein that is catalytically stimulated by the presence of nicked and unpaired DNA. Human RAP1 binds TRF2 and the yeast orthologue (yRAP1) binds yeast telomeric repeats and is involved in transcriptional silencing Morse, R., 2000 Trends Genet. 16, 51-53). Both PARP and RAP1 have been implicated in the recruitment of DNA polymerase to replication origins (Dantzer, F. et al, 1998 Nucl. Acids Res., 26:1891-1898) and to the replication of the C-rich strand of eukaryotic telomeres (Diede, S. J. and D. E. Gottschling, 1999 Cell, 99:723-733). Additionally, PARP is important in the checkpoint response to DNA damage (Galande, S., and T. Kohwi-Shigematsu, 1999 J. Biol. Chem., 274:20521-20528) and it is possible that these telomere binding proteins may be important for downregulating EBV replication and maintenance in response to genotoxic stress.

Based on these findings, the inventor has determined that EBNA1 utilizes a novel mechanism to recruit DNA polymerase and maintain genome copy number. The inventor's results indicate that pharmacological agents acting on poly-ADP ribosylase and telomerase interfere with EBV latency, and therefore are useful as therapies for EBV associated diseases. Additionally several other human herpesviruses have similar binding sites for telomere binding factors. Interference with these enzymes (PARP, tankyrase and telomerase) is useful in the treatment of HSV, CMV, VZV, HHV6-8 latency.

The following examples illustrate several embodiments of this invention. These examples are illustrative only, and do not limit the scope of the present invention.

### EXAMPLE 1

### Purification and Identification of Cellular Proteins Recruited to oriP by EBNA1

To identify cellular proteins that mediate EBNA1 functions at OriP, a stable HeLa cell derivative (SL1) was established expressing epitope tagged EBNA1 (HA-EBNA1) and episomal OriP. SL1 cells were generated by retroviral transformation of HeLa S3 cells with HA-EBNA1 lacking Gly-Ala repeats (Δ102-325). HeLa and SL1 cell nuclear extracts were prepared by the method of Dignam, J. D. et al, 1983 Nucl. Acids Res. 11, 1475-1489. Nuclear pellets were solubilized by sonication in RIPA buffer (1% Triton×100, 0.1% sodium deoxycholate, 140 mM NaCl, 50 mM Hepes (pH 7.5), 1 mM EDTA, 1 mM DTT, and protease inhibitors-1 mM PMSF, 1 mM benzamidine, 10 μg/ml aprotinin, 20 μg/ml leupeptine, 1 μg/ml pepstatin, 25 μg/ml TLCK and 50 μg/ml TPCK). Sonicated nuclear pellets were clarified by centrifugation at 39,000×g for 30 minutes. This solubilized nuclear pellets were combined with nuclear extract (1:1) made 6 mM MgCl₂, and preincubated with 400 μg/ml sonicated herring sperm DNA for 30 minutes. Biotinylated PCR fragments were coupled to streptavidin conjugated magnetic bead (Dynal) and then incubated nuclear extracts for 45 minutes at 25° C. The bound material was washed 4 times in D150 buffer (20 mM HEPES, pH7.9, 20% glycerol, 0.2 mM EDTA, 150 mM KCl, 1 mM DTT, 1 mM PMSF) and then eluted with D1000 (same as above except 1000 mM KCl).

In one experiment this single round affinity purified material was assayed by Western. In another experiment, the nuclear extracts derived from SL1 (EBNA1⁺) or control HeLa cells were subject to a second round of DNA affinity purification by dilution to 150 mM KCl in the same buffer using the 130 bp DS region of OriP.

Silver staining of DS affinity purified proteins revealed several polypeptides derived from SL1 cells and specific to SL1 cell extract bound to control DNA (Z₇E4T promoter) or to Dyad symmetry DNA. Western blotting indicated that EBNA1 was abundant in the DS affinity purified material from SL1 cells. HA-EBNA1 was detected in SL1 cells affinity purified with DS element, but not with control Z₇E4T promoter DNA.

Using the above-described DNA affinity chromatography, several cellular EBNA1-dependent Dyad Symmetry (DS) interacting HeLa proteins were purified from HeLa or SL1 (HA-EBNA1 expressing HeLa) cells. These isolated proteins associated with DS element of EBV oriP in an EBNA1-dependent manner. Several of the more abundant polypeptides isolated by DNA affinity purification were identified using microcapillary reverse phase HPLC nano-electrospray tandem mass spectrometry (mLC/MS/MS) and were found to be members of a telomere associated protein complex:

(a) p60 is TRF-2, a myb DNA binding family member that regulates telomeric length (Broccoli, D. et al, 1997 Nat. Genet. 17, 231-235).

(b) p58 is a human RAP1, another myb family member related to TRF2 and the human orthologue of the budding yeast telomere repeat binding factor; RAP1 has been shown to interact with TRF2 in vitro and in vivo (Li, B. et al, 2000 Cell, 101:471-483).

(c) The largest polypeptide, p150, was identified as Tankyrase, a TRF1 binding protein possessing poly ADP ribosyltransferase activity and ankyrin repeat domains (Smith, S., et al, 1998 Science 282:1484-1487). Tankyrase associates with telomeres in vivo and binds to TRF1, a third member of the myb family of telomeric binding factors.

(d) p116 is human poly-ADP ribosylase PARP1 which has not previously been found at telomeres, but is functionally related to telomere-associated Tankyrase by virtue of their shared enzymatic activity (D'Amours, D. et al, 1999 Biochem. J., 342:249-268).

(e) p32 is human tat-associated protein p32, which had been found to associate with EBNA1 in two hybrids and immunoprecipitation assays (Wang, Y. et al, 1997 Virol., 236:18-29; and Van Scoy, S. et al, 2000 Virol. 275, 145-157).

(f) p50 was identified as a fragment of p58, i.e., TRF2-interacting protein RAP1.

(g) The 54 kD band was confirmed to be EBNA1.

Several other polypeptides were identified as EBNA1 breakdown products (p45 and 14) and are assumed to be proteolytic fragments, suggesting that some proteolysis of EBNA1 may have occurred during the isolation procedure. Table 1 below reports the MS/MS spectra data.

TABLE 1

| | | Genbank Id | Spectra |
|---|---|---|---|
| Polypeptide | Name | No. | MS/MS |
| p150 | Tankyrase | 4507613 | 48 |
| p116 | PARP | 627553 | 35 |
| p60 | TRF2 | 2529440 | 23 |
| p58 | TRF2-interacting telomeric RAP1 | 8102033 | 7 |
| p54 | EBNA1 | | |
| p45 | EBNA1 | | |
| p32 | tat-associated protein p32 | 1096067 | 10 |
| p14 | EBNA1 | | |

### EXAMPLE 2

### Western Analysis of DS Element Affinity Purified Factors

To confirm the identity of proteins identified by mLC/MS/MS and demonstrate that EBNA1 stabilizes the recruitment of telomere-associated proteins at OriP DS, the DS affinity purified, DS associated polypeptides identified by MS/MS were analyzed by Western blotting antibodies specific for Tankyrase and TRF2 (IMGENEX), TRF1 (Santa Cruz Biotech), PARP (Trevigen), EBNA1 or anti-HA.

The initial purification scheme involved two rounds of DNA affinity chromatography, which was required for high quality MS/MS analysis. Western blotting experiments were performed with HeLa or SL1 nuclear extract material generated by single round DNA affinity purifications washed either at low stringency (150 mM KCl) or at high stringency (300 mM KCl). The starting material from HeLa or SL1 represented 5% of the starting binding reaction.

To determine if these proteins specifically bound the DS element, SL1 extract was affinity purified over a single round of DS or with control DNA of similar size and assayed by Western blotting with these antibodies except that the antibodies were overlayed on a single blot, The Western blot analysis of SL1 extracts were purified over control DNA Z₇E4T or DS element.

Tankyrase was highly enriched in DS-affinity purified preparations derived from SL1 cells and was stable to 300 mM KCl salt washes. Tankyrase from the SL1 extracts bound DS only in the presence of EBNA1 and had no affinity for control DNA (Z₅E4t control promoter). TRF2 was also enriched in DS affinity preparations derived from SL1 cells, although significant binding occurred with HeLa control extracts washed with low salt. Interestingly TRF2 had weak affinity for DS in the absence of EBNA1,but its association with DS was highly stabilized by the presence of EBNA1. TRF2 did not bind to control DNA, indicating it recognizes specific sequences in DS. Antibodies to the TRF2-related protein, TRF1, revealed a weak signal in the input, but enrichment in DS affinity purified preparations from SL1 cells.

In contrast, PARP bound to DS equally well in HeLa and SL1 extracts in the presence or absence of EBNA1,but did not bind to control DNA, suggesting that EBNA1-dependent PARP binding occurs only after two rounds of affinity purification. However, based on silver staining described in Example 1, the evidence demonstrated that EBNA1 stabilizes PARP association after two rounds of DNA affinity chromatography.

As expected, EBNA1 bound only in the SL1 extract and to the DS, with no binding to control DNA.

These results indicate EBNA1 recruits a family of telomere associated proteins to DS; that Tankyrase and TRF1 bind to DS in an EBNA1-dependent manner, while TRF2 and PARP are stabilized by EBNA1. Tankyrase, PARP, TRF2, and EBNA1 bound exclusively to the DS element, and were not detectable in control DNA affinity preparations, indicating that these proteins specifically recognize the DS element.

### EXAMPLE 3

### Telomere Binding Proteins Interact with ORIP in RAJI Burkitt Lymphoma Cells; TRF2 and Tankyrase Bind ORIP in Vivo

To determine if telomere associated proteins EBNA1, TRF2 and Tankyrase associate with EBV oriP episomes in naturally infected cell lines, the chromatin immunoprecipitation) (CHIP) assay was used with EBV positive RAJI Burkitt lymphoma cell lines that contain ˜5-25 copies of episomal EBV. Chromatin immunoprecipitation was performed as described in Galande, S. & Kohwi-Shigematsu, 1999 J. Biol. Chem. 274, 20521-20528.

Briefly, asynchronous RAJI cells were cross-linked with formaldehyde, sonicated to generate chromatin DNA fragments of 400 bp average size, and then immunoprecipitation with various antibodies to FLAG-M2 (Sigma), EBNA1, Tankyrase, or TRF2. DNA fragments isolated from immunoprecipitation protein-DNA complexes (three-fold dilutions for each set of antibodies) were then amplified for 24 cycles by PCR with primer pairs specific for the DS region of EBV oriP (corresponding to EBV nucleotides 8586-9206, nucleotides 1-621 SEQ ID NO: 1), or for control EBV sequences surrounding the BRLF1 promoters (nucleotides 106,094-106,420, nucleotides 1-317 SEQ ID NO: 2), over 90 kb away from oriP and unlikely to share OriP binding proteins.

EBNA1 antibodies efficiently precipitated OriP sequence with no detectable BRLF1 amplification and control antibodies did not reveal significant immunoprecipitation of either fragment. Immunoprecipitation with Tankyrase and TRF2-specific antibodies revealed substantial precipitation of OriP DNA, relative to control BRLF1 sequence. Chromatin was precipitated with antibodies to TRF2, Tankyrase, EBNA1 or control antibody FLAG-M2. Input DNA titration indicated similar amplification efficiency of the DS and BRLF1. Control immunoprecipitation with anti-FLAG antibody revealed background oriP and no detectable BRLF1 sequence. Immunoprecipitation with EBNA1 antibodies revealed significant oriP DNA, and no detectable BRLF1 DNA. Similarly DNA amplified from TRF2 immunoprecipitates was highly specific for oriP, relative to BRLF1. Antibodies to Tankyrase also showed specific enrichment for oriP, although a significant amount of control BRLF1 DNA was amplified in the highest concentration. Quantitation from the average of two experiments is shown in the panel below.

Quantitative analysis of amplified DNA fragments indicated that both Tankyrase and TRF2 specifically associates with OriP. The lower efficiency of TRF2 binding to OriP relative to EBNA1 may be expected since EBNA1 can bind as a stable dimer to 25 distinct sites in the entire OriP, while TRF2 may only bind intermittently to 3 sites in the DS. The low efficiency of Tankyrase immunoprecipitation may result from its indirect binding to DNA. Nevertheless, these results indicate that TRF2 and Tankyrase can be specifically associated with OriP sequence on episomal EBV genomes in vivo.

### EXAMPLE 4

### EBNA1 Stimulates TRF2 Binding to the DS Nonamer Repeats

TRF2 binds the TTAGGG nonamer repeat elements in human telomeres (Broccoli D. et al, 1997 Nat. Genet., 17:231-235). TTAGGG is found three times in DS and has been characterized as the core of the nonomer repeat element (Yates, J. L., et al, 2000 J. Virol., 74:4512-4522). These three repeat sequences resemble telomeric repeat elements (TTAGGGTT) (Vogel, M. et al, 1998 BioTechniques 24, 540-544). Genomic footprinting revealed cell cycled dependent changes at the nonomer, and ligation mediated PCR indicated that new synthesis of viral DNA initiates at the nonomer element in oriP (Niller, H. H. et al, 1995 BioTechniques, 270:12864-12868). Mutagenesis of the three nonomers reduced DS function from a minimal DS replicator in replication assays and inhibited OriP-dependent plasmid maintenance in plasmid maintenance assays (Vogel, M. et al, 1998 BioTechniques, 24:540-544 and Yates et al, cited above). The nonamers are the site of a cell cycle-dependent change in the genomic footprint and the origin of DNA strand synthesis based on ligation-mediated PCR analysis of OriP. These results suggest that the nonamers interact with cellular proteins important for the modulation of OriP-dependent DNA replication and plasmid maintenance.

TRF2, which binds telomeric repeat DNA, was assayed for its ability to bind the nonamer sequences found in the DS region of OriP by DNase I footprinting. Recombinant EBNA1 and TRF2 generated from baculovirus were assayed by DNase I footprinting, of the OriP DS region. TRF2 (500 ng) and EBNA1 (15 ng) were compared alone and together for DNase footprinting on the antisense or sense strand. Wild type (wt) or nonamer substitution mutant were used as probes for DNase I footprinting for TRF2 over a concentration range 12 to 500 ng (three fold dilutions) in the absence or presence of EBNA1 (50 ng).

The DNase I Footpriniting assay is performed as follow: The DS sequence was ³²P radiolabelled by Klenow fill-in of the Asp718 site (antisense) or the Xho I site (sense) of DS-GL2 Pro (Promega derivative) and DNase footprinting was performed as described in Rawlins, D. R. et al, 1995 Cell 42, 859-868. The nonamer mutant DS (Δa, b, c) was generated by site directed mutagenesis (Stratagene Quickchange Kit) with 6 bp substitutions in each of the three nonamer sites. EBNA1 and TRF2 were expressed and purified from baculovirus infected SF9 cells.

EBNA1 binds to four sites in DS and its binding induced strong DNase I hypersensitive sites in all three nonamer repeat sequences. Addition of TRF2 protected and altered the EBNA1 hypersensitive sites overlapping the nonamer repeats. TRF2 did not produce strong footprints over these sites in the absence of EBNA1, indicating that EBNA1 stimulates TRF2 binding to the nonamer sites. A DS mutant with substitutions in all three nonamers was tested for TRF2 binding in the presence and absence of EBNA1 by DNase I footprinting. With wild type DS, TRF2 bound nonamer site a and b in the presence of EBNA1, but not in its absence. With the nonamer mutant template, TRF2 had no effect on the DNase I footprint pattern in the absence or presence of EBNA1. These results demonstrate that the nonamer sequences, which have been genetically linked to OriP replication and plasmid maintenance functions are required for TRF2 binding.

Sequences protected by EBNA1 and TRF2 in DNase I footprinting assays are illustrated below:

| TRF2₃ EBNA1₄ EBNA1₃ | |
|---|---|
| ← | |
| AACCCTAATTCGATAGCATATGCTTCCCGTTGGGTAACATATGCTATTGAA | (SEQ ID NO: 3) |
| \| | |
| 9023 | |
| TRF2_{b} EBNA1₂ EBNA1₁ | |
| → | |
| TTAGGGTTAGTCTGGATAGTATATACTACTACCCGGGAAGCATATGCTAC | |
| \| \| | |
| 9073 9086 | |
| TRF2_{c} | |
| → | |
| CCGTTTAGGGTTA | |
| \| | |
| 9126 | |

### EXAMPLE 5

### Potential Role for NAD-Dependent Regulation of EBNA1-ORIP Complex Formation

Both PARP and Tankyrase are predicted to have poly ACP ribose polymerase activity. To determine whether singly round DS affinity purified proteins had PARP activity, Dyad symmetry affinity purified proteins from HeLa or SL1 extracts were incubated with 0.1 mM ³²P-NAD in a ribosylation assay. ³²P-NAD-dependent polyADP-ribose polymerase reactions with purified PARP in the absence or presence of activating sonicated salmon sperm DNA and histone substrates. Reactions were supplemented with DS element DNA or with histones and sonciated salmon sperm DNA.

Commercial preparations of homogeneous PARP had little activity in the absence of sonicated DNA. However, additional of sonicated DNA strongly stimulated auto-ribosylase activity and ribosylation of histone H1. DS affinity purified material from Hela or SL, cells were both found to have signification PARP activity in the absence of sonicated DNA, suggesting DS associates with PARP activity in the absence of EBNA1. However, the SL extract generated a novel ribosylated protein of 54 kD. Addition of DS DNA to the reaction had little effect, perhaps increasing ribosylation of a high molecular weight species not easily resolved in this experiment. The results suggest that DS associated complexes possess PARP activity and that EBNA1 induces ribosylation of a novel 54 kD substrate. Since EBNA1 is 54 kD, EBNA1 is the likely substrate.

To determine the effect of ribosylation on the association of proteins with DS, DNA affinity chromatography was performed in the presence or absence of 1 mM NAD. The addition of NAD was found to strong inhibit the association of EBNA1 and several EBNA1 associated proteins with DS. NAD did not inhibit the association of several nonspecific binding proteins, or substoichiometric polypeptides not identified by MS/MS. These results suggest that NAD induced ribosylation inhibits EBNA1 complex formation on oriP, and further suggest that these components may be important for the negative regulation of EBNA1 function at oriP.

These new findings demonstrate that telomeric binding proteins associate with oriP DS element and modulate EBNA1 function. These proteins may participate in the recruitment of DNA polymerase and DNA replication initiation function, or have other functions in plasmid maintenance as well.

### EXAMPLE 6

### TRF1 and TRF2 Affect DS Replication Function

A. Dominant negative truncation mutants of TRF2 and TRF1 have been used to reveal their function in telomere length maintenance (Smogorzewska, A. et al., 2000 Mol. Cell. Biol. 20, 1659-1568). OriP-dependent DNA replication was measured by transient transfection assay in which replicated plasmids are distinguished from non-replicated plasmids by resistance to the methylation-specific restriction enzyme Dpn1. A DNA replication assay was performed in 293 cells to assay truncation mutants of TRF1 and TRF2 for their effect on OriP-dependent DNA replication essentially as described in Yates, J. L. et al, 2000 J. Virol. 74, 4512-4522. ΔTRF1 (aa72-439) and ΔTRF2 (aa 45-454) were expressed in pCMV-FLAG-2 (Sigma). HA-EBNA1 was expressed in pcDLSRα296. Dpn1 resistant, OriP-containing pHEBO plasmid was assayed for transient DNA replication in transfected 293 cells. pHEBO was cotransfected with pHA-EBNA1 or with control vector and with either ΔTRF1, ΔTRF2 or CMV2-FLAG vector. Dpn1 resistant DNA linearized with BamHI was used as one control. BamHI divested pHEBO (1 and 0.1 ng) was used as another control. pHEBO specific probes were generated by random priming for Southern blots. HA-EBNA1 expression levels in transfected cells was assayed by Western blotting with anti-HA. Dpn1-resistant pHEBO DNA observed in the top panel of A was quantitated by phosphorimager analysis.

OriP containing plasmid (pHEBO) was detected in 293 cells cotransfected with EBNA1, but was not detected in the absence of EBNA1, indicating that replication was strictly EBNA1-dependent. Cotransfection of deletion mutants of TRF1 and TRF2 reduced Dpn1 resistant pHEBO DNA to ˜30% of the amount found with control vector. Control Bam HI digestion revealed that plasmids were isolated with equal efficiency from all transfected cells. Expression levels of EBNA1 protein did not vary significantly, and was therefore not responsible for the different levels of OriP replication. TRF1 and TRF2 truncation proteins were expressed at high levels as determined by Western blotting.

These results indicate that overexpression of TRF1 and TRF2 truncation mutants interferes with efficient replication of OriP, and further support a role for TRF1 and/or TRF2 in OriP-dependent replication in vivo.

B. In another experiment, a deletion of TRF2 found to be dominant negative for telomere function (van Steensel, B. et al, 1998 Cell, 92:401-413) was generated and assayed for its effect on EBNA1 transcriptional activation of DS using a luciferase based reporter system in HeLa cells. The plasmid Dyad-GL2-luc contains a single copy of the Dyad element upstream of the SV40 minimal promoter driving luciferase. Full length TRF2 (1-500) or Δ TRF2 (Δ45-454) were expressed in pCMV-FLAG-2 (Sigma).

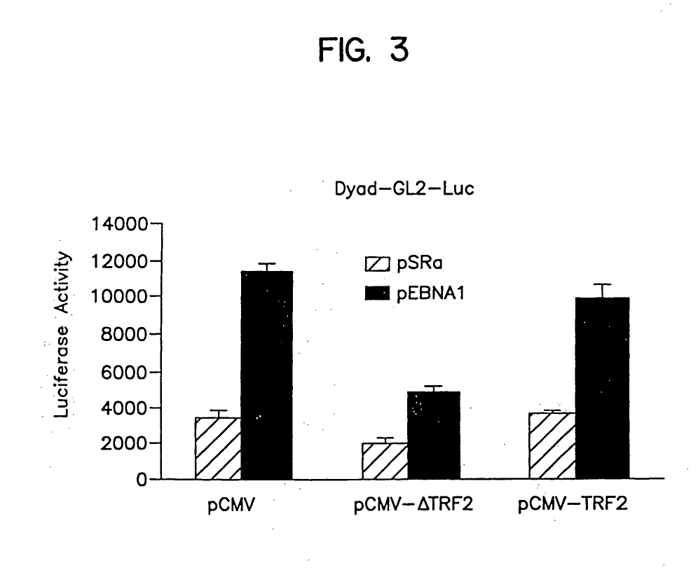

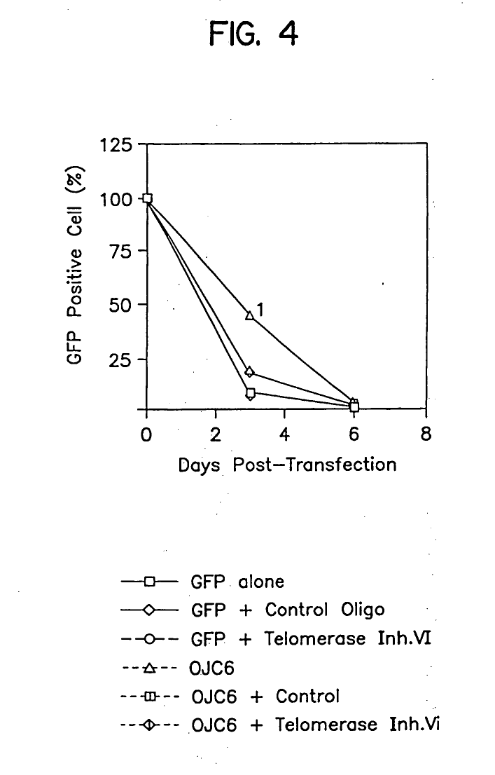

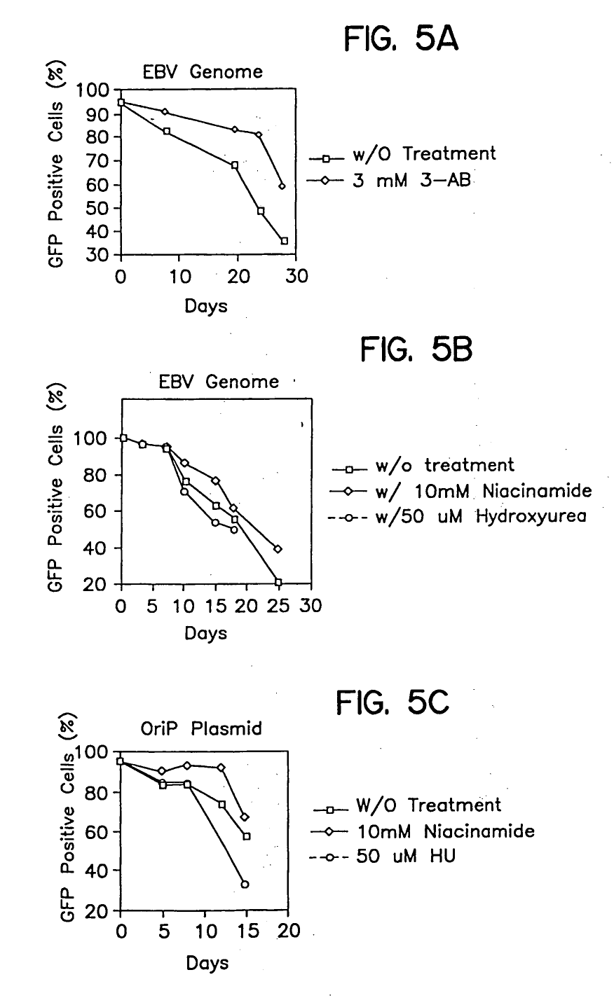

Cotransfection of the TRF2 dominant negative deletion mutant inhibited the basal activity, i.e., Dyad dependent transcription and the EBNA1stimulated transcription from DS in transient transfection assays. See FIG. 3. In contrast, TRF2 full length protein had no inhibitory effect on EBNA1 or DS mediated transcription levels. These results also demonstrate that TRF2 is important for EBNA1 transcription function at DS in vivo.

### EXAMPLE 7

### Screening Assay for Compounds Affecting EBV Plasmid Maintenance Function

A green fluorescent protein (GFP) reporter gene is inserted into the polylinker site of plasmid pREP10 (Invitrogen), which contains the EBV oriP and EBNA-1 under the control of its weak, naturally-occurring viral promoter. The GFP reporter gene allows for rapid flow cytometric (FACS) analysis to determine the percent of cells carrying the DNA vector. FACS is also used to cell sort, i.e., to obtain a homogeneous population of cells all expressing GFP and therefore carrying the vector.

The recombinant episomal plasmid GFP-REP10 is transfected into recipient mammalian or avian host cells and cells are sorted after 48 hours to obtain a homogeneous population (100%) expressing the GFP label. Test compounds, individually or in combination, or controls are applied to samples of the culture medium to test for the effect on GFP positive cells. Cells treated with test compounds, or with positive and/or negative controls, are assayed for the rate of loss of GFP over several days to weeks. These rates of loss are compared for each test compound with the relevant controls to determine novel test compounds that enhance or decrease episomal maintenance in the cell.

Confirmation of episomal DNA is determined at the experimental endpoint of between 3 to 5 weeks, using the HIRT extraction and Southern blotting procedure, described in e.g., Yates et al, 2000, J. Virol. 74, 4512-4522.

### EXAMPLE 8

### Additional Studies

A. Role of the Nonamer Repeats in Response to Pharmacological Modulation of OriP-Plasmid Maintenance.

In one study, D98/HR1 cells were transfected with OriP wt or OriPΔa,b,c, GFP-selected by FACS, and cultured in the presence of niacinamide (PARP inhibitor), hydroxyurea (PARP stimulator), or 3-AB (PARP inhibitor). OriP maintenance was assayed 3 weeks after sorting by Southern analysis of Hirt lysates.

The results of this study showed that niacinamide and 3-AB enhanced plasmid maintenance was dependent upon the nonamer binding sites in OriP.

B. Correlation Between poly-ADP Ribosylation of DS-Binding Proteins and OriP Plasmid Maintenance.

Dyad Symmetry (DS) affinity purified proteins from nuclear extracts derived from untreated hydroxyurea, or niacinamide-treated D98/HR1 cells were assayed by Western blot with antibodies specific to poly-ADP-ribose (αPAR) modification or anti-EBNA1. Changes in poly-ADP ribosylated protein isomers correlated with changes in OriP maintenance observed in the results of study A.

C. TRF1 and hRap1 Bind to DS in a Nonamer-Dependent Manner.

Raji nuclear extracts were purified by DNA affinity chromatography with wildtype DS (DSwt) or DS Δa, b, c. The affinity purified proteins were assayed by Western blot with newly generated antibodies specific for TRF1 and hRap1.The results of this study indicated that TRF1, hRap1, Tankyrase, and TRF2 bind to DS in a nonamer-dependent manner, and can be isolated from latently infected Raji cell extracts.

D. TRF1 and hRap1 Bind to OriP in Raji Burkitt Lymphoma Cells in Vivo.

A chromatin immunoprecipitation (ChIP) assay demonstrate that hRap1 and TRF1 are specifically associated with OriP DNA, but not with BZLF1promoter DNA in, vivo.

E. TRF1 Binding is Reduced by Hydroxyurea Treatment of D98/HR1Cells.

EBNA1, hRap1,and TRF1 association with OriP were determined by ChIP assay in cells that were treated with hydroxyurea or niacinamide. The results indicated that hydroxyurea treatment reduced TRF1 association, but not EBNA1 or hRap1. PARP modification of TRF1 causes a loss of TRF1 binding, and a decreased efficiency of plasmid maintenance.

F. TRF1 Binding is Reduced by H₂O₂ Treatment D98/HR1 Cells.

EBNA1, hRap1, and TRF1 were assayed for OriP association using the ChIP assay in D98/cells treated with H₂O₂(a known agonist of PARP) or niacinamide (inhibitor of PARP). TRF1 binding was reduced by H₂O₂ treatments suggesting that PARP-modified TRF1 dissociates from OriP. This result further supports the role of genotoxic agents regulating OriP function through modification of nonamer-binding proteins.

G. Modulation of MCM3 Binding in Response to Drug Treatment.

EBNA1 and MCM3 binding to DS were assayed in Akata cells cultured with no treatment, with hydroxyurea, or with niacinamide. Treatment of Akata cells with hydroxyurea let to a decrease in EBNA1 and MCM3 association, while treatment with niacinamide increased the MCM3 binding relative to EBNA1. These data suggest that PARP modifications may regulate MCM complex binding to OriP. Future quantification of these preliminary results will be necessary to determine the significance of MCM3 and other MCM complex components interaction with OriP.

H. Changes in Histone H3 Modification Correlate with Changes in Episomal Maintenance.

Akata cells were cultured without treatment, with H₂O₂, hydroxyurea (HU) or niacinamide as indicated above each lane. OriP-associated histone modification was measured by ChIP assay with antibodies specific for Flag, Acetylated H3, acetylated H4, or S10 phosphorylated H3. H₂O₂ inhibited H3 acetylation and S10 phosphorylation, while HU inhibited S10 phosphorylation of histone H3. These results suggest that histone modifications correlate with PARP-modification. These results also suggest that histone modifications may play a role in episome stability and plasmid maintenance.

Numerous modifications and variations of the present invention are included in the above-identified specification and are expected to be obvious to one of skill in the art. Such modifications and alterations to the compositions and processes of the present invention are believed to be encompassed in the scope of the claims appended hereto. All references are incorporated by reference herein.

1. A method for identifying a compound useful in the treatment of an infection by an episomal DNA virus, said method comprising the steps of:
providing a culture of mammalian or avian cells containing multiple copies of a recombinant viral extrachromosomal episome having an episomal maintenance element, said episomes being associated with a detectable label;
contacting said culture with a test molecule that inhibits the interaction between the episomal maintenance element on said episomes and at least one enzyme selected from a family of enzymes consisting of the poly-ADP ribose polymerase (PARP) family, the poly-ADP glycosylase (PARG) family, the telomerase family, the telomere repeat binding protein (TRF) family, and the myb DNA binding family that interferes with telomeres; and
detecting the elimination of said episomes from said cells by detecting the presence of said detectable label in the medium of said culture.

2. The method according to claim 1, wherein said virus is a member of the Herpesviridae family.

3. The method according to claim 2, wherein said virus is selected from the group consisting of Herpes Simplex virus (HSV), human Herpes virus 6 (HHV6), human Herpes virus 7 (HHV7), human Herpes virus 8 (HHV8), Cytomegalovirus (CMV), Epstein Barr virus (EBV), and Varicella zoster virus (VZV).

4. The method according to claim 1, wherein said virus is a papillomavirus.

5. The method according to claim 1, wherein said virus is the causative agent of Marek's disease.

6. The method according to claim 1, wherein said episomal maintenance element is a segment of viral DNA involved in maintaining the virus in a cell.

7. The method according to claim 6, wherein said episomal maintenance element is selected from the group consisting of a viral origin of replication, a terminal repeat sequence, an autonomous replicating sequence and a matrix attachment sequence.

8. The method according to claim 1, wherein said cell is an epithelial cell, a ganglion, a monocyte, an endothelial cell, and a lymphocyte

9. The method according to claim 1, wherein said enzyme is a member of the TRF selected from the group consisting of telomere repeat binding protein 1, telomere repeat binding protein 2, and human RAP1.

10. The method according to claim 1, wherein said enzyme is Tankyrase, a member of tie PARP family.

11. The method according to claim 1, wherein said contacting step employs more than one test molecule.

12. The method according to claim 1, wherein said contacting step inhibits the association of the episomal maintenance element with more than one of said enzymes.

13. The method according to claim 1, further comprising the step of assessing said test compound for toxicity to said cells.

14. The method according to claim 1, wherein said test molecule is a protein or peptide.

15. The method according to claim 1, wherein said molecule is a small chemical molecule.

16. The method according to claim 14, wherein said protein is an antibody.

17. The method according to claim 1, wherein said test molecule inhibits the association by binding to one or more of said enzymes.

18. The method according to claim 1, wherein said test molecule inhibits the association by preventing the binding of one or more of said enzymes to the viral origin of replication.

19. The method according to claim 1, further comprising the step of exposing said cells to an agent of genotoxic stress or an agent that inhibits DNA repair.

20. A kit for use in an assay for identifying a compound useful in the treatment of infections by episomal DNA virus, said kit comprising:
a culture of mammalian or avian cells containing multiple copies of a recombinant viral extrachromosomal episome having an episomal maintenance element, said episome being associated with a detectable label;
means for detecting the presence of said detectable label in the medium of said culture following contact with a test molecule; and
instructions for performing said assay.

21. A pharmaceutical or veterinary composition comprising in a physiologically acceptable carrier, at least one compound that inhibits the interaction between the episomal maintenance element of a viral episome residing in the cells of a mammalian subject previously infected with an episomal DNA virus and at least one enzyme selected from a family of enzymes consisting of the poly-ADP ribose polymerase family, the poly-ADP glycosylase family, the telomerase family, the myb DNA binding family that interferes with telomeres, and the telomere repeat binding protein family.

22. The composition according to claim 21, wherein said virus is a member of the Herpesviridae family.

23. The composition according to claim 22, wherein said virus is selected from the group consisting of Herpes Simplex virus, human Herpes virus 6, human Herpes virus 7, human Herpes virus 8, Cytomegalovirus, Epstein Barr virus, and Varicella zoster virus.

24. The composition according to claim 21, wherein said virus is a papillomavirus.

25. The composition according to claim 21, wherein said virus is the causative agent of Marek's disease.

26. The composition according to claim 21, wherein said episomal maintenance element is a segment of viral DNA involved in maintaining the virus in a cell.

27. The composition according to claim 21, wherein said compound is identified by a method comprising the steps of:
providing a culture of mammalian or avian cells containing multiple copies of a recombinant viral extrachromosomal episome having an episomal maintenance element, said episome being associated with a detectable label;
contacting said culture with said compound; and
detecting the elimination of said episomes from said cells by detecting the presence of said detectable label in the medium of said culture.

28. The composition according to claim 21, further comprising a DNA damaging agent or an agent that inhibits DNA repair.

29. A method for treating an infection by an episomal DNA virus comprising the step of:
administering to a mammalian subject having cells carrying multiple copies of a recombinant viral extrachromosomal episomes having an episomal maintenance element an effective amount of at least one compound that inhibits the interaction between the episomal maintenance element and at least one enzyme selected from the group consisting of a family of enzymes consisting of the poly-ADP ribose polymerase family, the poly-ADP glycosylase family, the telomerase family, the myb DNA binding family that interferes with telomeres, and the telomere repeat binding protein family.

30. The method according to claim 29, further comprising administering to said subject a DNA damaging agent or an inhibitor of DNA repair.

31. A method of increasing the cellular stability of a DNA molecule comprising an episomal maintenance element of an episomal DNA virus, said method comprising contacting said cell with an effective amount of a compound that inhibits the enzymatic activity of a member of the poly-ADP ribose polymerase enzyme family in the cell.

32. The method according to claim 31, wherein said DNA molecule is a recombinant, non-naturally occurring molecule.

33. The method according to claim 31, wherein said molecule is a gene therapy vector.


SEQUENCE LISTING (3 sequences)

SEQ ID NO: 1 - DNA, 621 bp, Epstein-Barr virus
gagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggccc

SEQ ID NO: 2 - DNA, 317 bp, Epstein-Barr virus
atacaaataaatttctcttacctgcgtctgtttgtgtagtgaggtgttgtgtcctgtatggtattctactttaaaaaggccggctgacatggattactggtcttttatgagccattggcatgggcgggacaatcgcaatataaaaccctgaccatcacatggggcattaggcgactctgcatcagcatcgcttaagtatgagtgggcagcagagaggctcggttattttggttcctgaacatctggctggggcattaactaagcttatgagcgattttatcacaggacaagatgtcactctttctggaggaaatatt

SEQ ID NO: 3 - DNA, 114 bp, Epstein-Barr virus
aaccctaattcgatagcatatgcttcccgttgggtaacatatgctattgaattagggttagtctggatagtatatactactacccgggaagcatatgctacccgtttagggtta